(19) United States
(12) Patent Application Publication
(10) Pub. No.: US 2002/0088019 A1
(43) Pub. Date: Jul. 4, 2002

(54) Methods of and pharmaceutical compositions for improving implantation of embryos

(76) Inventor: Oron Yacoby-Zeevi, Meitar (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(21) Appl. No.: 09/978,297

(22) Filed: Oct. 17, 2001

Related U.S. Application Data
(63) Continuation-in-part of application No. 09/260,037, filed on Mar. 2, 1999.
Application No. 09/260,037 is a continuation-in-part of application No. 09/140,888, filed on Aug. 27, 1998.
Application No. 09/140,888 is a continuation-in-part of application No. 09/046,475, filed on Mar. 25, 1998.
Application No. 09/046,475 is a continuation-in-part of application No. 08/922,170, filed on Sep. 2, 1997.
(60) Provisional application No. 60/240,037, filed on Oct. 16, 2000.

(51) Int. Cl. A61K 38/47

(52) U.S. Cl. 800/21; 424/94.61

(57) ABSTRACT

Methods of improving embryo implantation are disclosed and comprise contacting a receptive uterus and/or an embryo with an effective amount of heparanase and implanting the embryo in the receptive uterus.

This is a continuation-in-part of U.S. patent application Ser. No. 09/260,037, filed Mar. 2, 1999, which is a continuation-in-part of U.S. patent application No. Ser. 09/140,888, filed Aug. 27, 1998, which is a continuation-in-part of U.S. patent application Ser. No. 09/046,475, filed Mar. 25, 1998, now, U.S. Pat. No. 6,153,187, issued Nov. 28, 2000, which is a continuation-in-part of U.S. Pat. application No. 08/922,170, filed Sep. 2, 1997, now U.S. Pat. No. 5,968,822, issued Oct. 19, 1999. This application further claims the benefit of priority from U.S. Provisional Patent Application No. 60/240,037, filed Oct. 17, 2000. The specifications of the above cited applications are incorporated herein by reference.

### FIELD AND BACKGROUND OF THE INVENTION

The present invention relates to methods and pharmaceutical compositions for improving embryos implantation rate and, more particularly, to methods and pharmaceutical compositions for improving in vitro fertilization embryos implantation rate.

Infertility and Assisted Reproductive Technologies:

Treatment of infertility problems is a growing area of health care. Approximately 50,000 human in vitro fertilization (IVF) procedures are performed in the United States annually. Although costs vary widely depending on drugs, testing and other laboratory fees, typical IVF charges are on the order of $ 10,000 per procedure, not counting travel, lost work time, and emotional costs for the patients. Additionally, in treatment of animals, the ability to control the birth of offspring is important for agricultural concerns and for preservation of endangered species.

Many approaches are being developed to resolve problems with infertility. Infertility is understood to be the inability to conceive after six to twelve months of sexual activity without the use of contraceptives, depending on the age of the persons involved. Because infertility exerts extreme physical, emotional and financial stresses on those who are unable to conceive, there is a great need for improved aids for reproduction. These aids are typically referred to as Assisted Reproductive Technologies (ART).

In vitro Fertilization (IVF) followed by Embryo Transplantation:

By far the most common ART component is IVF, which has grown explosively in the two decades since it was developed. In its simplest form, IVF consists of pharmaceutical stimulation of the female's ovaries to produce a large number of follicles. Eggs surgically harvested from these follicles are then mixed in the laboratory with the male's sperm. If fertilization is successful, the embryos are incubated for a short time and then transferred back to the female. If one of these embryos implants in the uterine wall, a successful pregnancy may follow.

There are several modifications of this basic technique. For example, intracytoplasmic sperm injection (ICSI) can be used for cases of low sperm count or cases where the sperm has difficulty fertilizing the egg. Another IVF modification is Assisted Hatching (AH), a procedure in which the zona pellucida (the outer wall of the embryo) is mechanically cut or chemically etched, thereby partially exposing the embryo. In some laboratories, this procedure significantly improves implantation rate, particularly for older patients. Finally, IVF procedures can also incorporate donor tissues, including sperm, ova and embryos, for those individuals who cannot produce their own.

Proteoglycans (PGs):

Proteoglycans (previously named mucopolysaccharides) are remarkably complex molecules and are found in every tissue of the body. They are associated with each other and also with the other major structural components such as collagen and elastin. Some PGs interact with certain adhesive proteins, such as fibronectin and laminin. The long extended nature of the polysaccharide chains of PGs, the glycosaminoglycans (GAGs), and their ability to gel, allow relatively free diffusion of small molecules, but restrict the passage of large macromolecules. Because of their extended structures and the huge macromolecular aggregates they often form, they occupy a large volume of the extracellular matrix relative to proteins. Murry R K and Keeley F W; Biochemistry, Ch. 57. pp. 667-85.

Heparan Sulfate Proteoglycans (HSPGs):

HSPGs are acidic polysaccharide-protein conjugates associated with cell membranes and extracellular matrices. HSPGs bind avidly to a variety of biologic effector molecules, including extracellular matrix components, growth factor, growth factor binding proteins, cytokines, cell adhesion molecules, proteins of lipid metabolism, degradative enzymes, and protease inhibitors. Owing to these interactions, HSPGs play a dynamic role in biology, in fact most functions of the proteoglycans are attributable to the heparan sulfate (HS) chains, contributing to cell-cell interactions and cell growth and differentiation in a number of systems. HS maintains tissue integrity and endothelial cell function. It serves as an adhesion molecule and presents adhesion-inducing cytokines (especially chemokines), facilitating localization and activation of leukocytes. HS modulates the activation and the action of enzymes secreted by inflammatory cells. The function of HS changes during the course of the immune response are due to changes in the metabolism of HS and to the differential expression of and competition between HS-binding molecules. Selvan RS et al; Ann. NY Acad. Sci. 1996, 797: 127-39.

HSPGs are also prominent components of blood vessels (Wight TN et al; Arteriosclerosis, 1989, 9: 1-20). In large vessels HSPGs are concentrated mostly in the intima and inner media, whereas in capillaries HSPGs are found mainly in the subendothelial basement membrane, where they support proliferating and migrating endothelial cells and stabilize the structure of the capillary wall. The ability of HSPGs to interact with extracellular matrix (ECM) macromolecules such as collagen, laminin and fibronectin, and with different attachment sites on plasma membranes suggests a key role for this proteoglycan in the self-assembly and insolubility of ECM components, as well as in cell adhesion and locomotion.

Heparanase—a GAGs Degrading Enzyme:

Degradation of GAGs is carried out by a battery of lysosomal hydrolases. One important enzyme involved in the catabolism of certain GAGs is heparanase. It is an endo-β-glucuronidase that cleaves heparan sulfate at specific interchain sites.

The enzymatic degradation of glycosaminoglycans is reviewed By Ernst et al. (Critical Reviews in Biochemistry and Molecular Biology, 30(5):387-444 (1995). The common feature of GAGs structure is repeated disaccharide units consisting of a uronic acid and hexosamine. Various GAGs differ in the composition of the disaccharide units and in type and level of modifications, such as C5-epimerization and N or O-sulfation. Sulfated GAGs include heparin, heparan sulfate condroitin sulfate, dermatan sulfate and keratan sulfate. Heparan sulfate and heparin are composed of repeated units of glucosamine and glucuronic/iduronic acid, which undergo modifications such as C5-epimerization, N-sulfation and O-sulfation. Heparin is characterized by a higher level of modifications than heparan sulfate.

GAGs can be depolymerized enzymatically either by eliminative cleavage with lyases (EC 4.2.2.-) or by hydrolytic cleavage with hydrolases (EC 3.2.1.-). Often, these enzymes are specific for residues in the polysaccharide chain with certain modifications. GAGs degrading lyases are mainly of bacterial origin. In the eliminative cleavage, C5 hydrogen of uronic acid is abstracted, forming an unsaturated C4-5 bond, whereas in the hydrolytic mechanism a proton is donated to the glycosidic oxygen and creating an O5 oxonium ion followed by water addition which neutralizes the oxonium ion and saturates all carbons (Lindhart et al. 1986, Appl. Biochem. Biotech. 12:135-75). The lyases can only cleave linkages on the non-reducing side of the of uronic acids, as the carboxylic group of uronic acid participates in the reaction. The hydrolyses, on the other hand, can be specific for either of the two bonds in the repeating disaccharides. In pages 414 and 424 of the review, tables 8 and 14, Ernst et al. list the known GAG degrading enzymes. These tables describe substrate specificity, cleavage mechanism, cleavage linkage, product length and mode of action (endo/exolytic). Heparanase is defined as a GAG hydrolase which cleaves heparin and heparan sulfate at the β1,4 linkage between glucuronic acid and glucosamine. Heparanase is an endolytic enzyme and the average product length is 8-12 saccharides. The other known heparin/heparan sulfate degrading enzymes are β-glucuronidase, α-L iduronidase and α-N acetylglucosaminidase which are exolytic enzymes, each one cleaves a specific linkage within the polysaccharide chain and generates disaccharides. In table 8 the authors list two heparanases; platelet heparanase and tumor heparanase, which share the same substrate and mechanism of action. These two were later on found to be identical at the molecular level (Freeman et al. Biochem J. (1999) 342, 361-268, Vlodavsky et al. Nat. Med. 5(7):793-802, 1999, Hullet et al. Nature Medicine 5(7):803-809, 1999).

Heparin and heparan sulfate fragments generated via heparanase catalyzed hydrolysis are inherently characterized by saturated non-reducing ends, derivatives of N-acetyl-glucoseamin. The reducing sugar of heparin or heparan sulfate fragments generated by heparanase hydrolysis contain a hydroxyl group at carbon 4 and it is therefore UV inactive at 232 nm.

Interaction of T and B lymphocytes, platelets, granulocytes, macrophages and mast cells with the subendothelial extracellular matrix (ECM) is associated with degradation of heparan sulfate by heparanase activity. The enzyme is released from intracellular compartments (e.g., lysosomes, specific granules) in response to various activation signals (e.g., thrombin, calcium ionophore, immune complexes, antigens and mitogens), suggesting its regulated involvement in inflammation and cellular immunity. Vlodavsky I et al; Invasion Metas. 1992; 12(2): 112-27. In contrast, various tumor cells appear to express and secrete heparanase in a constitutive manner in correlation with their metastatic potential. Nakajima M et al; J. Cell. Biochem. 1988 Feb; 36(2):157-67. Important processes in the tissue invasion by leukocytes include their adhesion to the luminal surface of the vascular endothelium, their passage through the vascular endothelial cell layer and the subsequent degradation of the underlying basal lamina and extracellular matrix with a battery of secreted and/or cell surface protease and glycosidase activities. Cleavage of HS by heparanase may therefore result in disassembly of the subendothelial ECM and hence may play a decisive role in extravasation of normal and malignant blood-borne cells (Vlodavsky I et al; Inv. Metast. 1992, 12: 112-27, Vlodavsky I et al; Inv. Metast. 1995, 14: 290-302).

It has been previously demonstrated that heparanase may not only function in cell migration and invasion, but may also elicit an indirect neovascular response (Vlodavsky I et al; Trends Biochem. Sci. 1991, 16: 268-71). The ECM HSPGs provide a natural storage depot for bFGF. Heparanase mediated release of active bFGF from its storage within ECM may therefore provide a novel mechanism for induction of neovascularization in normal and pathological situations (Vlodavsky I et al; Cell. Molec. Aspects. 1993, Acad. Press. Inc. pp. 327-343, Thunberg L et al; FEBS Lett. 1980, 117: 203-6). Degradation of heparan sulfate by heparanase results in the release of other heparin-binding growth factors, as well as enzymes and plasma proteins that are sequestered by heparan sulfate in basement membranes, extracellular matrices and cell surfaces. Selvan RS et al; Ann. NY Acad. Sci. 1996, 797: 127-39.

Present Limitations of IVF:

Despite its great successes, IVF has several significant problems. First and foremost, the procedure is unpredictable. Although the ideal result of any IVF procedure is a single, live birth, a viable pregnancy occurs in only about 30% of all procedures. Conversely, IVF may result in a pregnancy with multiple embryos. In this regard, twins and triplets pose relatively few risks beyond a single embryo pregnancy. The potential for problems, however, increases for higher order births. Selective embryo reduction is therefore often recommended for these cases which increases the psychological trauma for the parents.

Like unassisted reproduction, IVF begins with a source of sperm and ova. There is a virtually 100% certainty of obtaining these materials, using donor tissues if necessary. Next, fertilization occurs, and good IVF laboratories typically have a fertilization success rate of about 75%, using ICSI if appropriate. After a short incubation period, the resulting embryo is then introduced into the uterus, where implantation occurs. Implantation is generally the limiting factor in overall IVF success.

Implantation itself, however, consists of several steps. First, the embryo must enter the uterine cavity. In normal reproduction, without ART, the embryo descends through the fallopian tubes. The embryo then comes into contact with some point on the uterine wall. Next, the embryo and wall surfaces fuse at the contact point. The uterine wall properties then change dramatically at the implantation site, thus allowing the embryo to become fully implanted. For IVF, the embryo is carried into the uterine cavity in a solution injected from a syringe inserted through the cervical canal.

Although the implantation process appears to be simple enough, it is actually quite complicated and requires the coordination of many factors, many of which are unknown. A failure of any one of these processes prevents implantation and thus pregnancy. Furthermore, it is believed that implantation failures may indeed be the reason that only about 20% of even the most fertile couples conceive in a given month of attempting pregnancy.

Improving the implantation rate would make the IVF process more reliable, more effective and yield many benefits. Improving the effectiveness of the IVF process above its current 30% would reduce the need for repeat procedures, a critical factor considering the costs and stresses involved for the patients. Another benefit is that an improved success rate would make better use of the quite limited supply of donor ova and embryos. Improving the success rate would also further extend the supply of donor tissues by enabling some patients, particularly those who are older but otherwise healthy, to use their own tissues. Such patients would be more than willing to leave the donor program given the opportunity to have children of their own genetic basis. Yet another benefit is that by reducing the number of embryos required for a successful procedure, the incentive to overstimulate the ovaries is reduced, thereby reducing possible harmful side effects to the patient. Finally, improved implantation rate would eliminate the incentive to transfer large numbers of embryos back to the uterus, thereby preventing multiple births.

These advantages have been noted before, resulting in a variety of efforts to improve the implantation process, including both chemical and mechanical methods. The chemical techniques employ both natural and artificial compounds to improve the conditions of the embryo, the uterus, or both.

Many of these chemical efforts employ pharmaceutical modification of the uterine surface to make it more receptive to the embryo. Although these efforts are promising, there is concern that any agent strong enough to alter the properties of the uterus may also harm the embryo, thereby leading to birth defects. Another approach to improve implantation involves incubating the embryos to the blastocyst or later stages before transfer. Although this approach is promising, one European group has recently established an Internet registry to track the possibility of related birth defects. At the present time, none of these methods are effective in increasing the success at a viable pregnancy.

Mechanical means have focused on assisted hatching (AH) and tissue cultures. AH involves eroding the wall of the embryo by chemical and/or physical attack so that the embryo can expand and attach more readily, a technique particularly useful for patients over 38 years of age. Tissue cultures of uterine wall cells promise a more hospitable environment while still in vitro, with the hope that these conditions will prevail after transfer.

None of these techniques has produced much success.

The Involvement of ECM and bFGF in Blastocyst Implantation:

At implantation, trophectoderm attaches to the apical uterine luminal epithelial cell surface. Molecular anatomy studies in humans and mice, and data from experimental models have identified several adhesion molecules that could take part in this process: integrins of the alpha v family, trophinin, CD44, cad-11, the H type I and Lewis y oligosaccharides and heparan sulfate. After attachment, interstitial trophoblast invasion occurs requiring a new repertoire of adhesive interactions with maternal ECM as well as stromal and vascular cell populations. Human anchorage sites contain columns of cytotrophoblasts in which self-attachment gives way progressively to adhesion to ECM and then interstitial migration. Aplin J D; Rev Reprod 1997, 2(2): 84-93. Lessey B A et is al; J Reprod Immuol 1998, 39(1-2): 105-16.

During the process of implantation in humans, fetal trophoblast cells invade and migrate into the maternal decidua. During this migration, trophoblast cells destroy the wall of the maternal spiral arteries, converting them from muscular vessels into flaccid sinusoidal sacs. This vascular transformation is important to ensure an adequate blood supply to the feto-placental unit. Both cell-cell and cell-matrix interactions are important for trophoblast invasion of the decidual stroma and decidual spiral arteries. Cell-matrix adhesions are mediated by specific receptors, mostly belonging to the family of integrins. Signals transduced to the cells from the matrix via integrins could play a pivotal role in the control of cellular behavior and gene expression, such as metalloproteinases that facilitate matrix degradation and tissue remodelling. Burrows TD et al; Hum Reprod Updat 1996, 2(4): 307-21. Thus, the trophoblastic cells of the blastocyst and of the placenta express an invasive phenotype. These cells produce and secrete metalloproteinases which are capable of digesting the extracellular matrix and invade it. Among the numerous endometrial factors that control trophoblastic invasion, the components of the ECM such as laminin and fibronectin, play an important role. The endometrial extracellular matrix is thus a potent regulator of trophoblast invasion. Bischof P et al; Contracept Fertil Sex 1994, 22(1): 48-52. The invasion of extravillous trophoblast cells into the maternal endometrium is one of the key events in human placentation. The ability of these cells to infiltrate the uterine wall and to anchor the placenta to it as well as their ability to infiltrate and to adjust utero-placental vessels to pregnancy depends, among other things, on their ability to secrete enzymes that degrade the extracellular matrix. Huppertz B et al; Cell Tissue Res. 1998, 291(1): 133-48.

Expression of the heparan sulfate proteoglycan, perlecan, on the external trophectdermal cell surfaces of mouse blastocysts increases during acquisition of attachment competence. Smith SE et al., Dev. Biol. 1997, 184(1): 38-47. Radioautographic data indicates that mouse decidual cells produce and secrete collagen and sulfated proteoglycans. Abrahamsohn P A et al; J. Exp. Zool. 1993 266(6): 603-28.

HSPGs are an integral constituent of the placental and decidual ECM. Because this proteoglycan specifically interacts with various macromolecules in the ECM, its degradation may disassemble the matrix. Hence, in the case of the placenta, this may facilitate normal placentation and trophoblast invasion. Incubation of cytotrophoblasts in contact with ECM results in release of ECM-bound bFGF. It was, therefore, proposed, yet so far never validated, that a cytotrophoblastic heparanase facilitates placentation, through cytotrophoblast extravasation and localized neovascularization. Goshen R et al; Mol. Hum. Reprod. 1996, 2(9): 679-84; Dempsey, L. A. et al. Trends Biochem Sci. Aug. 25, 2000 (8):349-51.

Mammalian embryo implantation involves a series of complex interactions between maternal and embryonic cells. Uterine polypeptide growth factors may play critical roles in these cell interactions. bFGF is a member of a family of growth factors. This growth factor may be potentially important for the process of embryo implantation because it (i) is stored within the ECM and is thus easily available during embryo invasion; (ii) is a potent modulator of cell proliferation and differentiation; (iii) stimulates angiogenesis. Chai N et al; Dev. Biol. 1998, 198(1): 105-15. Relatively high concentrations of bFGF significantly enhance the rate of blastocyst attachment and of trophoblast spreading and promoted the expansion of the surface area of the implanting embryos. Keratinocyte growth factor (KGF) and bFGF derived form the endometrial cells exert paracrine effects on the process of implantation by stimulating trophoblast outgrowth through their cognate receptors. Taniguchi F et al; Mol. Reprod. Dev. 1998, 50(1): 54-62; Yoshida S; Nippon Sanka Fujinka Gaddai Zasshi 1996, 48(3): 170-6.

mRNAs encoding bFGF were detected in all stages of the ovine preimplantation embryo, although the relative abundance of this transcript decreased from the one cell to the blastocyst stage, suggesting that it may represent a maternal transcript in early sheep embryos. The expression of growth factors transcripts very early in mammalian development would predict that these molecules fulfil a necessary role(s) in supporting the progression of early embryos through the preimplantation interval. Watson A J et al; Biol Reprod. 1994, 50(4): 725-33.

The cellular distribution of bFGF was examined immunohistocheically in the rat uterus during early pregnancy (days 2-6). bFGF localized intracellularly in stromal and epithelial cells and within the ECM at days 2 and 3. It was distinctly evident at the apical surface of epithelial cells at days 4 and 5 of pregnancy. Concurrent with this apical localization, bFGF was present in the uterine luminal fluid, suggesting release of this growth factor from epithelial cells. Embryonic implantation was accompanied by increased intracellular bFGF content in luminal epithelial and decidual cells. However, similar cells outside of the implantation site and in the artificially decidualized uterus did not express analogous bFGF levels, indicating that a unique signal from the embryo triggers bFGF expression. Changes in the cell-specific distribution of bFGF imply a multifunctional role for this growth factor in uterine cell proliferation, differentiation, and embryonic implantation. In addition, the apical release of bFGF from epithelial cells indicates utilization of a novel secretory pathway for bFGF export during early pregnancy. Carlone D L, Rider V; Biol. Rerod. 1993, 49(4): 653-65. In the mouse, FGF signaling induces the cell division of embryonic and extraembryonic cells in the preimplantation embryo starting at the fifth cell division. Chai N et al; Dev Biol 1998, 198(1): 105-15. bFGF is present within the implantation chamber on days 6-9 of pregnancy and may be involved in the decidual cell response, trophoblast cell invasion and angiogenesis. Wordinger R J et al; Growth factors. 1994, 11(3): 175-86.

It has been hypothesized for some time that secretions of the oviduct and uterus are involved in stimulating cell proliferation in preimplantation mammalian embryos and promotion of early differentiation events that lead to successful implantation. At least some of the regulatory factors present within uterine secretions are growth factors that can act along a paracrine pathway by binding to specific receptors on embryonic cells. Perhaps, then, in addition to functions of growth factors acting singly on their specific receptors, combinations of factors are important for induction of a specific developmental response. It is also possible that the result of combinations of factors may involve a process of interference whereby exposure of embryonic cells to one growth factor may compromise its ability to bind and respond to another.

Schulz G A, Heyner S; Oxf. Rev. Reprod. Biol. 1993, 15: 43-81.

Expression of Heparanase DNA in Animal Cells:

Stably transfected CHO cells expressed the heparanase gene products in a constitutive and stable manner. Several CHO cellular clones have been particularly productive in expressing heparanases, as determined by protein blot analysis and by activity assays. Although the heparanase DNA encodes for a large 543 amino acids protein (expected molecular weight about 65 kDa) the results clearly demonstrate the existence of two proteins, one of about 60-68 kDa and another of about 45-50 kDa. It has been previously shown that a 45-50 kDa protein with heparanase activity was isolated from placenta, Goshen, R. et al. Mol. Human Reprod. 1996, 2: 679-684, and from platelets, Freeman and Parish Biochem. J. 1998, 339:1341-1350. It is thus likely that the 65 kDa protein is the pro-enzyme, which is naturally processed in the host cell to yield the 45 kDa protein. The p50 was found to be active and the p65 protein was not active, further suggesting that the p50 is the active enzyme, and the p65 is a pro-enzyme.

Heparanase Assists in Introducing Biological Material into Patients:

PCT/US00/03353, which is incorporated herein by reference, teaches that when externally added, heparanase adheres to cells. Cells to which heparanase is externally adhered to process the heparanase to an active form. Cells to which an active form of heparanase is externally adhered protect the adhered heparanase from the surrounding medium, such that the adhered heparanase retains its catalytic activity under conditions which otherwise hamper its activity. Cells to which an active form of heparanase is externally adhered, either cells genetically modified to express and extracellularly present or secrete heparanase, or cells to which purified heparanase has been externally added, are much more readily translocatable within the body of experimental animal models, as compared to cells devoid of externally adhered heparanase. Inactive pro-heparanase can be processed by endogenous proteases into its active form, once adhered to cells. Hence, heparanase can be used to assist in introduction of biological materials, such as cells and tissues into desired locations in the bodies of patients.

Further details pertaining to heparanase, heparanase gene and their uses can be found in, for example, PCT/US99/09256; PCT/US98/17954; PCT/US99/09255; PCT/US99/25451; PCT/IL00/00358; PCT/US99/15643; PCT/US00/03542; and PCT/US99/06189; and in U.S. Pat. Nos. 6,242,238; 5,968,822; 6,153,187; 6,177,545; and 6,190,875, the contents of which are hereby incorporated by reference.

There is a widely recognized need for, and it would be highly advantageous to have, a method with which to increase embryo implantation success rate.

### SUMMARY OF THE INVENTION

According to one aspect of the present invention there is provided a method of improving embryo implantation, the method comprising contacting an embryo with an effective amount of heparanase and implanting the embryo in a receptive uterus.

According to another aspect of the present invention there is provided a method of improving embryo implantation, the method comprising contacting a receptive uterus with an effective amount of heparanase and implanting the embryo in the receptive uterus.

According to yet another aspect of the present invention there is provided a method of improving embryo implantation, the method comprising contacting a receptive uterus with an effective amount of heparanase, contacting an embryo with an effective amount of heparanase and implanting the embryo in the receptive uterus.

According to further features in preferred embodiments of the invention described below, contacting the embryo with an effective amount of heparanase is in vitro.

According to still further features in the described preferred embodiments contacting the embryo with an effective amount of heparanase is in utero.

According to still further features in the described preferred embodiments the embryo is generated in vitro via in vitro fertilization (IVF).

According to still further features in the described preferred embodiments contacting the receptive uterus with the effective amount of heparanase precedes implanting the embryo in the receptive uterus.

According to still further features in the described preferred embodiments contacting the receptive uterus with the effective amount of heparanase is concurrent to implanting the embryo in the receptive uterus.

According to still another aspect of the present invention there is provided an embryo of, for example, 4-30 cells, immersed in a solution containing, and/or coated with, exogenous heparanase.

According to an additional aspect of the present invention there is provided a pharmaceutical composition comprising, as an active ingredient, an effective amount of heparanase, the pharmaceutical composition is designed for intra-uterine application.

According to yet an additional aspect of the present invention there is provided a pharmaceutical composition comprising, as an active ingredient, an effective amount of heparanase, the pharmaceutical composition is designed for application to an embryo in vitro.

According to still an additional aspect of the present invention there is provided an embryo growth composition comprising an effective amount of nutrients for embryonic growth and an effective amount of heparanase for assisting in embryo implantation.

According to further features in preferred embodiments of the invention described below, the heparanase is a mature heparanase.

According to still further features in the described preferred embodiments the heparanase is a pro-heparanase, cleavable into mature heparanase.

The present invention successfully addresses the shortcomings of the presently known configurations by providing methods and compositions which increase the probability of embryo implantation.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 demonstrates the effect of heparanase on embryo implantation in mice. Pseudopregnant ICR mice were divided into 3 groups, Group 1 mice were implanted with untreated blastocysts (o, control), Group 2 mice were implanted with heparanase treated blastocysts (x, embryo), Group 3 mice were implanted with untreated blastocysts following intrauterine injection of 5 μl of heparanase (4 μg heparanase per uterine horn) (□, uterus). On day 9 of pregnancy ICR female mice were sacrificed by cervical dislocation and the number of fetuses were counted. The implantation rate was calculated as: [The number of embryos found in the uterus on day 9]×100=[Percent of the number of blastocysts transferred to the uterus implantation].

### DESCRIPTION OF THE PREFERRED EMBODIMENTS

The present invention is of methods and pharmaceutical compositions which can be used to improve embryos implantation rate. Specifically, the present invention can be used to improve in vitro fertilization embryos implantation rate. Improving embryo implantation rate would make the IVF process more reliable, more effective and yield many benefits. Improving the effectiveness of the IVF process above its current 30% would reduce the need for repeat procedures, a critical factor considering the costs and stresses involved for the patients. Another benefit is that an improved success rate would make better use of the quite limited supply of donor ova and embryos. Improving the success rate would also further extend the supply of donor tissues by enabling some patients, particularly those who are older but otherwise healthy, to use their own tissues. Such patients would be more than willing to leave the donor program given the opportunity to have children of their own genetic basis. Yet another benefit is that by reducing the number of embryos required for a successful procedure, the incentive to overstimulate the ovaries is reduced, thereby reducing possible harmful side effects to the patient. Finally, improved implantation rate would eliminate the incentive to transfer large numbers of embryos back to the uterus, thereby preventing multiple births. Other embryo implantation procedures, such as the procedures employed for reproducing endangered species (i.e., cross-species embryo implantation) and those implantation procedures used in other veterinary applications will also benefit the advantages offered by the present invention.

The principles and operation of the methods and pharmaceutical compositions according to the present invention may be better understood with reference to the descriptions that follows and the examples below.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

While mating heparanase overexpressing trangenic mice, it was unexpectedly noted that larger litters as compared to non-transgenic mice of a similar genetic background were obtained. This unexpected finding triggered a study aimed at determining whether heparanase can be used to improve embryo implantation rate. In one aspect of the study controlled mating of heparanase expressing transgenic and/or non-transgenic mice and careful monitoring of litter size was employed. In another aspect of the study, the effect of preimplantation exposure of embryos or uterus to heparanase on implantation success rate was monitored. In both cases, a clear indication was obtained, heparanase substantially improves embryo implantation rate.

According to one aspect of the present invention there is provided a method of improving embryo implantation. The method, according to this aspect of the invention, comprises contacting an embryo with an effective amount of heparanase and implanting the embryo in a receptive uterus.

As used herein, the term “embryo” refers to the cell mass resulting from fertilization of an oocyte with a spermatocyte, either in vivo or in vitro, and which is suitable for implantation in a receptive uterus. For example, where in vivo, fertilization is accomplished either by conventional intercourse or artificial insemination, where in vitro, fertilization is accomplished either by contact with sperm or sperm cells or via intracytoplasmic sperm injection (ICSI). An embryo which is useful for implantation according to preferred embodiments of the present invention includes various stages between a two-cell embryo and a blastocyst.

As used herein the phrase “receptive uterus” refers to a uterus of a female mammal that is ready for embryo implantation. In humans there are well established protocols for transforming a uterus into a receptive uterus. Further details can be found in “A textbook of in vitro fertilization and assisted reproduction: the Bourn Hall Guide to clinical and laboratory practice”, Peter R Brindsen; or in the “Handbook of IVF”, Trounson Alan & Gardner D K. In animals, more information can be found in the “Applied animal reproduction”, H. Joe kbearden, John W Fuquay; and in the “Manipulating the Mouse Embryo —A Laboratory Manual”, Hogan Beddington & Costantini Lacy.

As used herein the term “heparanase” refers to an animal endoglycosidase hydrolyzing enzyme which is specific for heparin or heparan sulfate proteoglycan substrates, as opposed to the activity of bacterial enzymes (heparinase I, II and III) which degrade heparin or heparan sulfate by means of β-elimination. The heparanase can be natural (purified) or recombinant and optionally modified, precursor (e.g., pro-heparanase or pre-pro-heparanase) or activated (e.g., mature heparanase) form, as described in, for example, PCT/US98/17954 and PCT/US99/09256, which are incorporated herein by reference.

For different applications, the concentration and source of heparanase used may vary. Depending on incubation time, heparanase at concentrations of 0.1 μg/ml to 1 mg/ml can be employed. Presently preferred concentrations are 1 μg/ml to 100 μg/ml.

As used herein in the specification and in the claims section below, the term “purified” includes also enriched. Methods of purification/enrichment of heparanase are well known in the art. Examples are provided in U.S. Pat. application Ser. No. 09/071,618, filed May 1, 1998, in Goshe R et al. Mol. Human Reprod. 2, 679-684, 1996 and in WO91/02977, which are incorporated herein by reference.

As used herein the term “natural” refers to an enzyme of a natural origin.

As used herein the term “recombinant” refers to an enzyme produced via genetic engeneering techniques.

As used herein in the specification and in the claims section below, any enzyme, such as heparanase, refers both to the inactive pro-enzyme form and to its processed active form.

According to another aspect of the present invention there is provided a method of improving embryo implantation. The method, according to this aspect of the invention, comprises contacting a receptive uterus with an effective amount of heparanase and implanting the embryo in the receptive uterus.

According to yet another aspect of the present invention there is provided a method of improving embryo implantation. The method, according to this aspect of the invention, comprises contacting a receptive uterus with an effective amount of heparanase, contacting an embryo with an effective amount of heparanase and implanting the embryo in the receptive uterus.

It will be appreciated that the present invention can also be used as a means of improving implantation success in a conventional conception process. To this end, intrauterine application of heparanase prior to or during conventional conception is envisaged.

According to preferred embodiments of the present invention, contacting the embryo with an effective amount of heparanase can be achieved in vitro, ex vivo, or in uterus.

According to preferred embodiments of the invention the embryo is generated in vitro via in vitro fertilization, is removed from a uterus of a donor female or is generated in the receptive uterus via conventional conception.

According to preferred embodiments of the invention contacting the receptive uterus with the effective amount of heparanase precedes implanting the embryo in the receptive uterus.

According to preferred embodiments of the invention contacting the receptive uterus with the effective amount of heparanase is concurrent to implanting the embryo in the receptive uterus.

According to another aspect of the present invention there is provided an embryo between 2 cells and a blastocyst, immersed in a solution containing, and/or coated with, exogenous heparanase.

As used herein the phrase “exogenous heparanase” means heparanase as this term is defined herein added beyond and over any amount of heparanase which may be present naturally.

According to an additional aspect of the present invention there is provided a pharmaceutical composition. The pharmaceutical composition according to this aspect of the present invention comprises, as an active ingredient, an effective amount of heparanase. The pharmaceutical composition according to this aspect of the present invention is designed for intrauterine application. As such, the pharmaceutical composition, comprise, in addition to heparanase, a carrier, typically, a saline solution, approved for intrauterus application. The pharmaceutical composition according to this aspect of the present invention may also include additional active ingredients, such as, but not limited to, leukocyte inibitory factor (LIF). In a preferred embodiment, the pharmaceutical composition according to this aspect of the present invention is contained in a container or dispenser adapted for intrauterus application. Such containers include an elongated neck or dispenser capable of reaching the uterus via the vagina and cervix. Preferably, the pharmaceutical composition according to this aspect of the present invention is identified for use in intravaginal application, in IVF procedures, and/or in embryo implantation procedures. Although local application is presently preferred, systemic (e.g., via injection or oral application) heparanase application may also be useful in implementing the present invention. The use of vaginal suppositories is also envisaged.

According to yet an additional aspect of the present invention there is provided a pharmaceutical composition. The pharmaceutical composition according to this aspect of the present invention comprises, as an active ingredient, an effective amount of heparanase. The pharmaceutical composition according to this aspect of the present invention is designed for application to an embryo in vitro. As such, the pharmaceutical composition, comprises, in addition to heparanase, a carrier, typically, a saline solution, approved for immersing embryos during IVF procedures. The pharmaceutical composition according to this aspect of the present invention may also include additional active ingredients, such as, but not limited to, LIF. In a preferred embodiment, the pharmaceutical composition according to this aspect of the present invention is contained in a disposable container. Preferably, the pharmaceutical composition according to this aspect of the present invention is identified for use in IVF procedures, and/or in embryo implantation procedures.

According to still an additional aspect of the present invention there is provided an embryo growth composition comprising an effective amount of nutrients for embryonic growth and an effective amount of heparanase for assisting in embryo implantation. Additional active ingredients may include LIF. The nutrients used are typically those included in IVF culture medium, as described in, for example, “A textbook of in vitro fertilization and assisted reproduction: the Bourn Hall Guide to clinical and laboratory practice”, Peter R Brindsen; or in the “Handbook of IVF”, Trounson Alan & Gardner D K.

The present invention successfully addresses the shortcomings of the presently known configurations by providing methods and compositions which increase the probability of successful embryo implantation.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### Reproduction of Heparanase Transgenic Mice

Transgemic Mice:

Trangenic mice carrying and expressing the human heparanase gene are described in U.S. patent application Ser. No. 09/864,321, which is incorporated herein by reference. The following describes the generation of these transgenic mice and some of their phenotypes.

High level constitutive expression of heparanase was driven by chicken beta-actin promoter. The plasmid pCAGGS (Niwa, H et al. Gene 108: 193-200, (1991) was modified to contain a unique EcoRI site at position 1719. An XbaI-EcoRI 1.7 kb fragment, which contained the entire open reading frame of human heparanase was cloned into the compatible sites of the vector. Before injection, the plasmid pCAGGS-hpa was digested with SalI and PstI in order to isolate the expression cassette and eliminate bacterial DNA sequences. The resulting fragment contained the CMV-IE enhancer, chicken β-actin promoter and hpa cDNA followed by a rabbit β-globin poly adenylation site.

The DNA fragment containing the hpa expression cassette was injected into fertilized eggs, derived from C57BL×BalbC breed. The isolation of fertilized eggs, injection of DNA and transplantation of blastocysts were conducted by the Department of cell biochemistry—the transgenic unit at the Hadassah Medical School, Jerusalem according to a protocol adapted from Hogan et al. Manipulating the Mouse Embryo A Laboratory Manual, Cold Spring Harbor Laboratory Press, 1994.

Mice developed from the injected blastocysts were tested for the presence of the human hpa transgene in their genome. Genomic DNA was extracted from tail tips of the mice and the human hpa transgene sequence was amplified using human hpa specific PCR primers. To this end, tail fragments were incubated overnight at 55° C. in a lysis buffer (8 M urea, 0.2 M Tris-HCl, 0.4 M NaCl, 20 mM EDTA, 1% N-Laurylsarcosine, 10 μg/ml proteinase K). The dissolved tissue underwent phenol extraction and ethanol precipitation, to obtain a highly purified genomic DNA.

The integration of the human heparanase cDNA in the mouse genome was verified by PCR using two sets of primers. The first couple was designed to amplify the 5′ region of the transgene. It included a β-actin promoter specific primer (designated 5′-pCAGGs) 5′-ATAGGCAGCTGACCTGA-3′(SEQ ID NO:1) and human hpa specific primer: (designated Hpl-300) 5′-TGACTTGAGATTGCCAGTAACTTC-3′(SEQ ID NO:2). The second primers set was designed to amplify the 3′ region of the transgene. It included a human hpa specific primer (designated Hpu-830) 5′-CTGTCCAACTCAATGGTCTAACTC-3′ (SEQ ID NO:3), and a primer specific to the plasmid derived 3′-untranslated region (designated 3′pCAGGS) 5′-TCTAGAGCCTCTGCTAACCA-3′ (SEQ ID NO:4); PCR conditions were as follows: 2 minutes at 95° C. followed by 33 cycles of 15 seconds at 95° C., 1 minute at 58° C. and 1 minute at 72° C.

Four G₀ founder mice were obtained, harboring the human hpa cDNA in their genome as revealed by a PCR reaction specific for the human hpa cDNA. Founders were mated with C57B1 mice to create F1 mice and those were mated among themselves to create F2 mice. Homozygous F2 mice from each Go line were identified by Southern blot analysis and a quantitative PCR assay. Homozygousity was verified by mating with C57B1 mice, where all the pups were positive heterozygous. All founder transgenic mice were back crossed with C57BL mice in order to establish C57B1 transgenic mice with a pure genetic background.

Expression of the human heparanase protein was demonstrated by Western blot analysis of tissue extracts derived from F 1 transgenic and control mice. Measurements of heparanase activity in tissue extracts revealed a much higher activity in the transgenic as compared to control mice in all tissues examined. Immunohistochemical staining of tissue sections revealed a high expression of the human heparanase protein in tissues derived from the transgenic mice, but not control mice.

The transgenic mice were fertile and showed no apparent signs of abnormality. Few phenotypic alterations were however noted. For example, the virgin transgenic mice develop lobular-alveoli structures in the mammary gland, a phenomenon that is characteristic of mammary glands of pregnant mice.

Transgenic Mice Reproduction Phenotype:

In order to test the possible role of heparanase in embryo implantation normal or transgenic mice bearing the human heparanase gene were mated and their litter size recorded. Transgenic and normal, 8 weeks old, C57BL mice were mated. The litter size per female and number of pregnancies per group were recorded. Two separate experiments were conducted. The results are summarized in Table 1 below.

TABLE 1

| | Exp. 1: | | Exp. 2: | Mean |
|---|---|---|---|---|
| Group F/M | litter size | Mean (No. Preg.) | litter size | (No. Preg.) |
| N/N | 6, 7, 4, 7, | 5.5 (9/10) | 6, 7, 7, 7, | 6.5 (6/6) |
| | 4, 6, 5, 4, 7 | | 5, 7 | |
| T/T(G1) | 12, 11, 11, | 11 (6/6) | 13, 7, 9, | 10.5 (6/6) |
| | 10, 14, 8 | | 10, 11, 13 | |
| T/T(G3) | 8, 7, 8 | 8 (3/4) | 6, 6, 5, 5, | 6 (6/6) |
| | | | 7, 8 | |
| N/T(H1) | 4, 6, 8, 7 | 6 (4/6) | | |
| N/T(H3) | 6, 7, 6 | 6 (3/3) | | |

N/N = normal females (F) X normal males (M)
T/T(G1) = transgenic G1 F X transgenic G1 M
T/T(G3) = transgenic G3 F X transgenic G3 M
N/T(G1) = normal F X transgenic G1 M
N/T(G3) = normal F X transgenic G3 M

### Example 2

### Quantitative Assessment of Murine Implantation following Treatment with Heparanase

Materials and Experimental Procedures:

Mice:

50 female and 17 male ICR (CD-1®) mice, about 8-12 weeks of age at study commencement and 50 female and 20 male CB₆F1 mice, about 6-8 weeks of age at study commencement were used. ICR mice were used to obtain pseudopregnant females, whereas CB₆F1 mice were used to obtain transplantable blastocysts. Test animals were kept under environmental controlled housing conditions throughout the entire study period and were maintained in accordance with Harlan Biotech Israel (HBI) approved Standard Operation Procedures (SOP's). At the termination of a three days acclimatization period, ICR female mice were individually identified by ear notching.

Heparanase:

CHO-p65 heparanase (1.693 mg/ml; Batch No. 11-1) was used in all experiments performed. CHO-p65 heparanase was prepared according to the the protocol described in WO 01/7297. The enzyme was diluted in DMEM +5 5% FCS, 1.5:100 (final heparanase concentration 25 ≅g/ml) for the treatment of blastocysts. The enzyme was diluted with distiled water for intauterine injection, 1:2.2, to receive a final concentration of 0.8 mg/ml heparanase.

Additional Materials:

Pregnant Mare's Serum (PMSG) (080K08021); and Human Chorionic Gonadotropin (hCG) (040K1223) were purchased from Sigma-Israel (Jerusalem, Israel). DMEM with 4.5 mg/D-Glucose (116270); and heat inactivated Fetal Calf Serum (515412) were from Beit Haemek (Kibbutz Beit Haemek, Israel).

Generation of Vasectomized ICR Male Mice:

ICR male mice were anesthesized by intraperitoneal injection of Avertine (0.5 ml/mouse). A small transverse skin incision was made at a point level with the top of the legs and then a similar size transverse incision in the body wall. Both testes could be reached through the one incision. The fat pad of one of the testes was pulled out and the testis, vas deferens and epididymis accompanied it. The vas deferens was cut at two locations, 4-5 mm apart, and the testis was placed back inside the body wall. The same steps were repeated for the other testis. The skin was then sutured. 14 days following vasectomy mice were ready for mating.

Generation of Pseudopregnant ICR Female Mice:

Pseudopregnant ICR mice were prepared by mating ICR female mice in natural estrus with vasectomized sterile ICR male mice, placing 3 females in a cage with one male. In the following morning the females were checked for a copulation plug. Female mice with no copulation plug were sacrificed.

Preparation of CB₆F1 Female Mice for Mating with CB₆F1 Male Mice to Produce Blastocysts for Implantation in Pseudopregnant ICR Female Mice:

Superovulated females CB₆F 1 were used to obtain a sufficient number of blastocysts. The females were subjected to intraperitoneal injection of 0.1 ml (5 IU) Pregnant Mare's Serum (PMSG) on 11 a. m. 48 hours later the same females were injected intraperitoneally with 0.1 ml (5 IU) of Human Chorionic Gonadotropin (hCG). Following administration of hCG, one female was placed in a cage with one stud CB₆F1 male. In the following morning the females were checked for a copulation plug (day 1 of pregnancy). Female mice with no copulation plug were sacrificed. The procedure were performed at least once a week, each time 10-20 CB₆F1 females mice were mated with the same 10-20 CB₆F1 male mice, until all females CB₆F1 mice (50) were used.

Collecting Embryos:

On day 3 of pregnancy the superovulated CB₆F1 female mice were sacrificed by dislocation and the abdominal cavity were opened. Following removal of uterus by cutting across the cervix and below the junction with the oviduct, the uterus were placed in a small volume of DMEM medium supplemented with 5% fetal calf serum. Each horn were flushed using 25 G hypodermic needle, inserted into the cut cervix, and a syringe with 1 ml of the above medium. A total of 288 embryos at a blastocyst stage were collected.

Blastocysts Treatment:

One third of the amount of blastocysts collected were placed in serum containing heparanase (25 μg/ml), two third of the blastocysts collected were placed in DMEM medium supplemented with 5% fetal calf serum. All blastocysts were incubate over night in a humidified atmosphere of 95% air and 5% CO₂ at 37° C.

Blastocysts Implantation:

Pseudopregnant ICR mice were divided into 3 groups, Group 1 mice were implanted with untreated blastocysts, Group 2 mice were implanted with heparanase treated blastocysts, Group 3 mice were implanted with untreated blastocysts following intrauterine injection of 5 μl of heparanase (4 μg heparanase per uterine horn). The intended experimental design is provided in Table 2 below (Some deviations are noted in Table 3 below):

TABLE 2

| Group | No. of | | |
|---|---|---|---|
| No. | animals | No. of blastocysts implanted | Heparanase treatment |
| 1 | n = 8 | 96 (12/animal) | None |
| 2 | n = 8 | 96 (12/animal) | Blastocysts |
| 3 | n = 8 | 96 (12/animal) | Uterus |

On day 4 of pseudopregnancy (4 days following mating with sterile ICR males), recipient ICR female mice (those female mice that developed a copulation plug one day post mating) were anesthetized by intraperitoneal injection of Avertine (0.5 ml/mouse). A small dorsal midline longitudinal incision was made in the skin. The incision was then slid to the left or right until it reached over the ovary and a small incision was made in the body wall. The fat pad of the ovary were picked up and the ovary, oviduct and uterus were pulled out through the body wall. Using a stereomicroscope a hole was made in the uterus a few mm down from the oviduct-uterus junction. A transfer pipette containing approximately 6 blastocysts was then inserted into the hole and the blastocysts were expelled into the uterus. The same steps were repeated for the other horn. The skin was then sutured. For group 3 mice, 5 μl of heparanase solution (4 μg) was injected into each uterine horn and 30 minutes thereafter the blastocysts were transferred thereto as described above.

Embryos Count:

Clinical evaluation of mice was performed once daily, five working days a week, throughout the study period. Animals were monitored for signs of illness or reaction to treatment, until study termination. No such signs were observed. Individual body weights of recipient animals were determined just prior to implantation, and at study termination on day 9 of pregnancy.

On day 9 of pregnancy ICR female mice were sacrificed by cervical dislocation and the number of fetuses were counted, any macroscopic abnormalities in the fetuses or uterus were recorded.

Experimental Results:

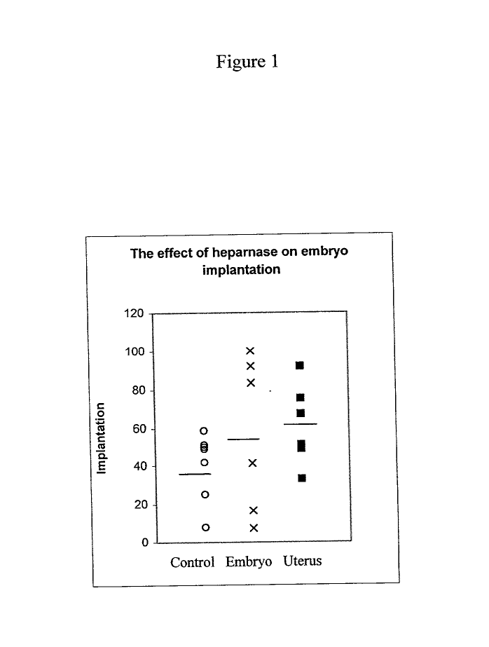

Table 3 and 4 below and FIG. 1 summarize the embryo count results.

TABLE 3

| | | Blastocysts Implantation | | Embryos | | | |
|---|---|---|---|---|---|---|---|
| | | | Number | | Number | | |
| Group No. | Treatment | Animal No. | per horn | Animal No. | per horn | | % |
| 1* | Control | 1 | 6 | 1 | R - 2 | L - 4 | 50 |
| | | 6 | 6 | 6 | R - 1 | L - 5 | 50 |
| | | 7 | 6 | 7 | R - 3 | L - 2 | 42 |
| | | 8 | 6 | 8 | R - 2 | L - 4 | 50 |
| | | 51 | 6 | 51 | R - 3 | L - 4 | 58 |
| | | 52 | 6 | 52 | R - 0 | L - 3 | 25 |
| | | 53 | 6 | 53 | R - 0 | L - 1 | 8 |
| 2** | Blastocysts | 9 | 6 | 9 | R - 5 | L - 5 | 83 |
| | treated with | 10 | 6 | 10 | R - 1 | L - 0 | 8 |
| | heparanase | 15 | 6 | 15 | R - 2 | L - 3 | 42 |
| | | 32 | 6 | 32 | R - 6 | L - 6 | 100 |
| | | 42 | 6 | 42 | R - 6 | L - 5 | 92 |
| | | 43 | 6 | 43 | R - 0 | L - 0 | 0 |
| | | 44 | R - 5, L - 6 | 44 | R - 0 | L - 2 | 17 |
| 3*** | Uterus | 17 | 6 | 17 | R - 3 | L - 5 | 67 |
| | treated with | 18 | 6 | 18 | R - 5 | L - 1 | 50 |
| | heparanase | 22 | R - 5, L - 7 | 22 | R - 1 | L - 3 | 33 |
| | | 23 | 6 | 23 | R - 1 | L - 5 | 50 |
| | | 36 | 6 | 36 | R - 2 | L - 7 | 75 |
| | | 38 | 6 | 38 | R - 6 | L - 5 | 92 |

TABLE 4

| | Mean No. of embryos | P value |
|---|---|---|
| | (% of implanted) | (compared to control) |
| Control | 40.5 | N.R. |
| Heparanase | 57 | 0.19 |
| treated blastocysts | | |
| Heparanase | 61 | 0.04 |
| treated uterus | | |

These results clearly demonstrate that heparanase increased the rate of implantation. The rate of implantation was significantly higher when heparanase was applied to the uterus prior to implantation (61% vs. 40.5%) and also higher when the blastocysts were treated with heparanase prior to implantation (57% vs. 40.5%).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations were apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of improving embryo implantation, the method comprising contacting an embryo with an effective amount of heparanase and implanting the embryo in a receptive uterus.

2. The method of claim 1, wherein said heparanase is a mature heparanase.

3. The method of claim 1, wherein said heparanase is a pro-heparanase, cleavable into mature heparanase.

4. The method of claim 1, wherein contacting the embryo with an effective amount of heparanase is in vitro.

5. The method of claim 1, wherein contacting the embryo with an effective amount of heparanase is in utero.

6. The method of claim 1, wherein the embryo is generated in vitro via in vitro fertilization.

7. A method of improving embryo implantation, the method comprising contacting a receptive uterus with an effective amount of heparanase and implanting the embryo in the receptive uterus.

8. The method of claim 7, wherein said heparanase is a mature heparanase.

9. The method of claim 7, wherein said heparanase is a pro-heparanase, cleavable into mature heparanase.

10. The method of claim 7, wherein the embryo is generated in vitro via in vitro fertilization.

11. The method of claim 7, wherein contacting the receptive uterus with the effective amount of heparanase precedes implanting the embryo in the receptive uterus.

12. The method of claim 7, wherein contacting the receptive uterus with the effective amount of heparanase is concurrent to implanting the embryo in the receptive uterus.

13. A method of improving embryo implantation, the method comprising contacting a receptive uterus with an effective amount of heparanase, contacting an embryo with an effective amount of heparanase and implanting the embryo in the receptive uterus.

14. The method of claim 13, wherein said heparanase is a mature heparanase.

15. The method of claim 13, wherein said heparanase is a pro-heparanase, cleavable into mature heparanase.

16. The method of claim 13, wherein contacting the embryo with an effective amount of heparanase is in vitro.

17. The method of claim 13, wherein contacting the embryo with an effective amount of heparanase is in utero.

18. The method of claim 13, wherein the embryo is generated in vitro via in vitro fertilization.

19. The method of claim 13, wherein contacting the receptive uterus with the effective amount of heparanase precedes implanting the embryo in the receptive uterus.

20. The method of claim 13, wherein contacting the receptive uterus with the effective amount of heparanase is concurrent to implanting the embryo in the receptive uterus.

21. A method of improving IVF embryo implantation, the method comprising contacting an IVF embryo with an effective amount of heparanase and implanting the embryo in a receptive uterus.

22. The method of claim 21, wherein said heparanase is a mature heparanase.

23. The method of claim 21, wherein said heparanase is a pro-heparanase, cleavable into mature heparanase.

24. The method of claim 21, wherein contacting the IVF embryo with an effective amount of heparanase is in vitro.

25. The method of claim 21, wherein contacting the IVF embryo with an effective amount of heparanase is in utero.

26. A method of improving IVF embryo implantation, the method comprising contacting a receptive uterus with an effective amount of heparanase and implanting the IVF embryo in the receptive uterus.

27. The method of claim 26, wherein said heparanase is a mature heparanase.

28. The method of claim 26, wherein said heparanase is a pro-heparanase, cleavable into mature heparanase.

29. The method of claim 26, wherein contacting the receptive uterus with the effective amount of heparanase precedes implanting the IVF embryo in the receptive uterus.

30. The method of claim 26, wherein contacting the receptive uterus with the effective amount of heparanase is concurrent to implanting the IVF embryo in the receptive uterus.

31. A method of improving IVF embryo implantation, the method comprising contacting a receptive uterus with an effective amount of heparanase, contacting an IVF embryo with an effective amount of heparanase and implanting the IVF embryo in the receptive uterus.

32. The method of claim 31, wherein said heparanase is a mature heparanase.

33. The method of claim 31, wherein said heparanase is a pro-heparanase, cleavable into mature heparanase.

34. The method of claim 31, wherein contacting the IVF embryo with an effective amount of heparanase is in vitro.

35. The method of claim 31, wherein contacting the IVF embryo with an effective amount of heparanase is in utero.

36. The method of claim 31, wherein contacting the receptive uterus with the effective amount of heparanase precedes implanting the IVF embryo in the receptive uterus.

37. The method of claim 31, wherein contacting the receptive uterus with the effective amount of heparanase is concurrent to implanting the IVF embryo in the receptive uterus.

38. An embryo coated with exogenous heparanase.

39. The embryo of claim 38, wherein said heparanase is a mature heparanase.

40. The embryo of claim 38, wherein said heparanase is a pro-heparanase, cleavable into mature heparanase.

41. The embryo of claim 38, between 2 cells and a blastocyst.

42. A pharmaceutical composition comprising, as an active ingredient, an effective amount of heparanase, the pharmaceutical composition is designed for intra-uterine application.

43. The pharmaceutical composition of claim 42, wherein said heparanase is a mature heparanase.

44. The pharmaceutical composition of claim 42, wherein said heparanase is a pro-heparanase, cleavable into mature heparanase.

45. A pharmaceutical composition comprising, as an active ingredient, an effective amount of heparanase, the pharmaceutical composition is designed for application to an embryo in vitro.

46. The pharmaceutical composition of claim 45, wherein said heparanase is a mature heparanase.

47. The pharmaceutical composition of claim 45, wherein said heparanase is a pro-heparanase, cleavable into mature heparanase.

48. An embryo growth composition comprising an effective amount of nutrients for embryonic growth and an effective amount of heparanase for assisting in embryo implantation.

49. The embryo growth composition of claim 48, wherein said heparanase is a mature heparanase.

50. The embryo growth composition of claim 48, wherein said heparanase is a pro-heparanase, cleavable into mature heparanase.


SEQUENCE LISTING (4 sequences)

SEQ ID NO: 1 - DNA, 17 bp, Artificial sequence
  synthetic oligonucleotide
ataggcagctgacctga

SEQ ID NO: 2 - DNA, 24 bp, Artificial sequence
  synthetic oligonucleotide
tgacttgagattgccagtaacttc

SEQ ID NO: 3 - DNA, 24 bp, Artificial sequence
  synthetic oligonucleotide
ctgtccaactcaatggtctaactc

SEQ ID NO: 4 - DNA, 20 bp, Artificial sequence
  synthetic oligonucleotide
tctagagcctctgctaacca